(19) United States
(12) Patent Application Publication
(10) Pub. No.: US 2026/0114828 A1
(43) Pub. Date: Apr. 30, 2026

(54) AUTOMATED BREAST DENSITY ASSESSMENT FOR FULL-FIELD DIGITAL MAMMOGRAPHY AND DIGITAL BREAST TOMOSYNTHESIS

(71) Applicant: Washington University, St. Louis, MO (US)

(72) Inventors: Shu Jiang, St. Louis, MO (US); Graham Colditz, St. Louis, MO (US)

(21) Appl. No.: 19/368,668

(22) Filed: Oct. 24, 2025

Related U.S. Application Data
(60) Provisional application No. 63/712,043, filed on Oct. 25, 2024.

(51) Int. Cl. A61B 6/50; A61B 6/00; A61B 6/02; G06N 3/045; G06N 3/096; G06T 7/00

(52) U.S. Cl. CPC A61B 6/502; A61B 6/025; A61B 6/5217; G06N 3/045; G06N 3/096; G06T 7/0012; G06T 2207/10112; G06T 2207/30068

(57) ABSTRACT

A computer implemented method of estimating tissue density of a body part of a subject from a medical image of tissue of the body part of the subject includes receiving at least one medical image of tissue of the body part of the subject, the received medical image being from a first imaging modality or a second imaging modality, estimating tissue density of the body part from the at least one medical image using a deep-learning model pretrained using images from the first imaging modality and images from the second imaging modality, and outputting an estimate of tissue density of the body part of the subject based on the tissue density estimated by the deep-learning model.

### CROSS REFERENCE TO RELATED APPLICATIONS

This application claims priority to U.S. Provisional Application Ser. No. 63/712,043, filed Oct. 25, 2024, which is hereby incorporated by reference in its entirety.

### STATEMENT REGARDING FEDERALLY SPONSORED RESEARCH & DEVELOPMENT

This invention was made with government support under CA246592, CA256810, and CA091842 awarded by the National Institutes of Health. The government has certain rights in the invention.

### BACKGROUND

This disclosure relates generally to systems and methods for automated assessment of breast density.

In the era of precision prevention and tailored screening, there is an increasing emphasis on getting the right prevention to the right person at the right time and tailoring screening examination protocols to people based on individual risk. Underpinning this approach is the need for accurate risk assessments that can be generated and delivered in real time in the clinic. With respect to women, strong evidence shows that adding mammographic density to breast cancer risk prediction models improves their performance. One systematic review identified 7 studies (out of 11) that showed significant increase in the area under the curve (AUC) when mammographic breast density was added to the prediction model. The increase in the AUC ranged from 0.03 to 0.14. Thus, major breast cancer risk models typically now include a measure of breast density, which is considered an intermediate marker of risk, as well as a surrogate endpoint in prevention trials.

There is a long record of epidemiologic investigations using mammographic density estimated from films and, more recently, from digital images. Additional research has focused on texture and other features beyond breast density. Current clinical mammographic density assessment relies heavily on subjective radiologist assessment as described in the 5th edition of the Breast Imaging Reporting and Data System (BI-RADS), rather than on quantitative volumetric analysis. However, the interobserver variability amongst radiologists is inevitable and occurs even with the same radiologist from year to year. This results in inconsistent and potentially less accurate recommendations for supplemental screening examinations such as MRI or ultrasound, and changes in the calculated risk assessment.

Accurate, efficient, and consistent processing of mammograms in real-time to guide subsequent clinical decisions is, therefore, a priority. Reporting of breast density to women as required by the US Food and Drug Administration (FDA) further drives the clinical need for accurate and consistent density assessment.

At least some known methods attempt to perform automated density estimation, mostly based on full-field digital mammography (FFDM) images. However, automated mammographic density assessment models have moved from using FFDM to digital breast tomosynthesis (DBT) in the USA. DBT improves the cancer detection rate on screening and has demonstrated usefulness in both screening and diagnostic settings. FFDM and DBT are both currently used in clinical practice, with over 80% of screening mammography now using DBT. At least one known system performs mammographic density estimation for both FFDM and DBT using raw mammogram data or “for processing” images. This data and images are typically not stored longer than a month in most clinics and research institutions as they not used for image interpretation. Thus, it is desirable that mammographic density estimation be automated using both FFDM and DBT images without reliance on raw mammogram data.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

One aspect of the present disclosure is a computer implemented method of estimating tissue density of a body part of a subject from a medical image of tissue of the body part of the subject. The method includes receiving at least one medical image of tissue of the body part of the subject, the received medical image being from a first imaging modality or a second imaging modality, estimating tissue density of the body part from the at least one medical image using a deep-learning model pretrained using images from the first imaging modality and images from the second imaging modality, and outputting an estimate of tissue density of the body part of the subject based on the tissue density estimated by the deep-learning model.

Another aspect of this disclosure is a system for estimating breast tissue density of a subject from a medical image of tissue of a breast of the subject includes a computing device having a processor and a memory. The memory stores instructions that cause the processor to: receive at least one medical image of tissue of the breast of the subject, the received medical image being an image from a first imaging modality or a second imaging modality, estimate breast tissue density from the at least one medical image using a deep-learning model pretrained using images from the first imaging modality and images from the second imaging modality, and output an estimate of the breast tissue density of the subject.

According to another aspect of this disclosure a method of estimating breast tissue density of a subject from at least one medical image of tissue of a breast of the subject includes receiving a plurality of first images acquired using a first imaging modality, the plurality of first images including at least one image of breast tissue for each first subject of a plurality of first subjects and a breast tissue density of each first subject having been previously determined, training a deep-learning model on the first plurality of images and the previously determined breast tissue density for each first subject, receiving a plurality of second images acquired using a second imaging modality, the plurality of second images including at least one image of breast tissue for each second subject of a plurality of second subjects and a breast tissue density of each second subject having been previously determined, further training the deep-learning model on the second plurality of images and the previously determined breast tissue density for each second subject, receiving at least one medical image of tissue of the breast of the subject, inputting the at least one medical image of tissue of the breast of the subject into the further trained deep-learning model, and receiving an estimated breast tissue density for the subject output by the further trained deep-learning model.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The following figures illustrate various aspects of the disclosure.

FIG. 1 is a simplified block diagram of an example system for estimating tissue density of a body part of a subject.

FIG. 2 is a flow diagram of an example method of estimating tissue density of a body part of a subject from a medical image of tissue of the body part of the subject according to the present disclosure.

FIG. 3 is a simplified block diagram of a computing device that may be used in the imaging systems of the present disclosure.

FIG. 4 is a graph of the distribution of the continuous density measures estimated from the FFDM using the proposed model in the external validation cohort.

FIG. 5 is a comparison of density estimation by a deep learning model according to the present disclosure and by radiologists reading FFDM.

FIG. 6 is a comparison of density estimation by a deep learning model according to the present disclosure and by radiologists reading DBT using synthetic DBT.

FIG. 7 is a comparison of density estimation for a cohort of women who were diagnosed with breast cancer, the comparison comparing estimation by a deep learning model according to the present disclosure and by radiologists reading FFDM.

FIG. 8 is a comparison of density estimation for a cohort of women who were diagnosed with breast cancer, the comparison comparing estimation by a deep learning model according to the present disclosure and by radiologists reading DBT using synthetic DBT.

Corresponding reference characters indicate corresponding parts throughout the drawings.

### DETAILED DESCRIPTION

This disclosure relates generally to systems and methods for automated assessment of tissue density. Example embodiments provide automated breast density assessment for two or more imaging modalities. The example embodiments will be described with respect to breast tissue density as the tissue density of interest and full-field digital mammography (FFDM) and digital breast tomosynthesis (DBT) as the imaging modalities, but the methods and systems described herein may be used in connection with any suitable tissue density of interest and images from any suitable imaging modalities.

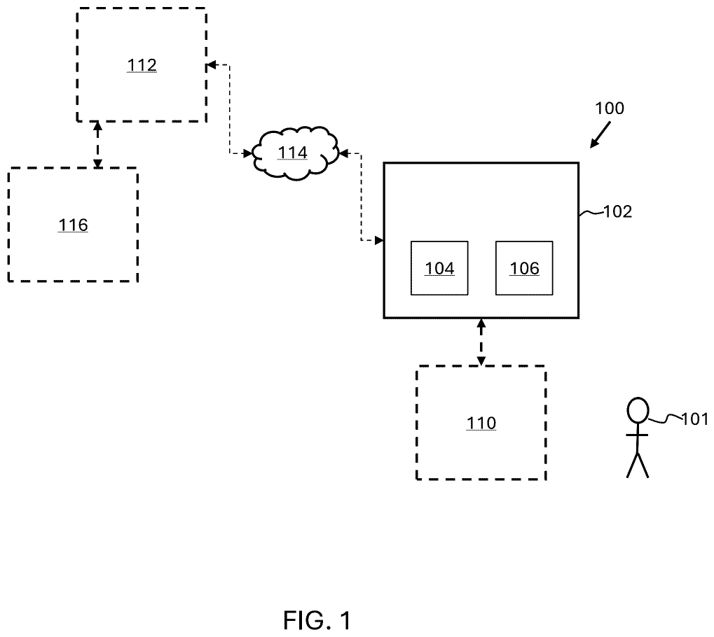

With reference to the figures, FIG. 1 is a system 100 for estimating breast tissue density of a subject 101 (who is not part of the system) from a medical image of tissue of a breast of the subject. The system includes a computing device 102 having a processor 104 and a memory 106. Example computing devices suitable for use as the computing device 102 will be described in detail below. The memory 106 stores instructions that cause the processor 104 to estimate tissue density of a body part of the subject 101 according to any of the methods described herein. Optionally, the system may include a medical imaging device 110 communicatively coupled to the computing device 102. The medical imaging device 110 may be collocated with the computing device 102 or may be remotely located from the computing device but communicatively coupled to the computing device. In some embodiments, the computing device 102 may control the medical imaging device 110 to acquire one or more medical images of the tissue of the body part of the subject 101. In other embodiments, the computing device 102 does not control the medical imaging device 110 but is still communicatively coupled to the imaging device to receive medical images generated by the medical imaging device. The system 100 may also optionally include a remote device 112 communicatively coupled to the computing device via a network 114 to provide medical image(s) of the tissue of the body part of the subject to the computing device 102. The remote device 112 may be a computer server, remote storage device, a remote computer device, or the like. The remote device 112 may store the medical images for transmission to or retrieval by the computing device 102 via the network 114. In some embodiments, the remote device 112 is coupled to a remote medical imaging device 116 that is operable to image the tissue of the body part of the subject 101, and the remote device may then provide or make available the medical image(s) to the computing device 102. In some embodiments, the computing device 102 may instruct/command/control/etc. the remote device 112.

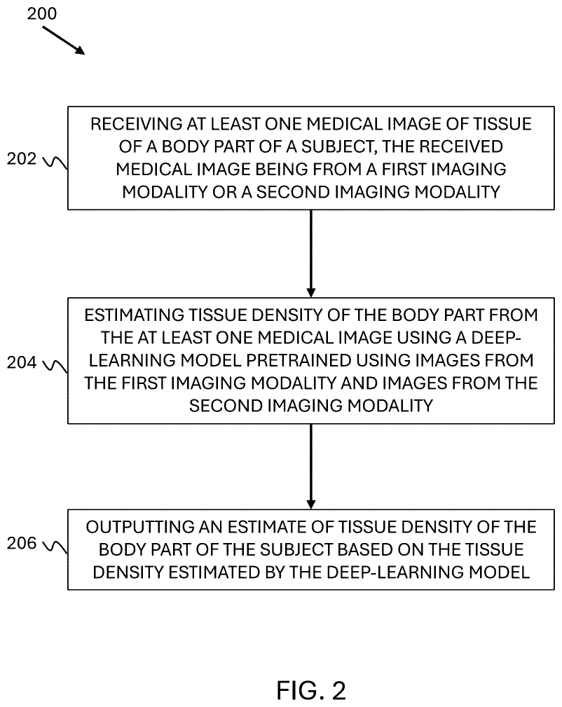

FIG. 2 is a flow diagram of an example computer implemented method 200 of estimating tissue density of a body part of a subject from a medical image of tissue of the body part of the subject according to the present disclosure. The method 200 will be described with respect to the system 100 but may be performed by any suitable system.

At 202, at least one medical image of tissue of the body part of the subject 101 is received by the computing device 102. The received medical image is from a first imaging modality or a second imaging modality. The at least one medical image may be received by the computing device 102 by being retrieved by the computing device from a remote or local storage device (e.g., remote device 112), by having the at least one medical image transmitted to the computer (such as from another computer 112, an imaging device 110, 116, etc.), by the computing device controlling an imaging device (e.g., medical imaging device 110 or 116) to generate the at least one medical image, or the like. In an example embodiment, the body part is a breast and the tissue density being estimated is breast tissue density. The at least one medical image is a plurality of medical images of the body part of the subject in various embodiments. For example, in some embodiments in which the body part is a breast, the at least one medical image is four images (also known as “views”), including craniocaudal [CC] and mediolateral-oblique [MLO] views. Other embodiments may receive any suitable number of views, such as two images, three images, five images, ten images, or the like. The received images are four views that are processed or “for presentation,” rather than the raw image data or “for process” images (which as noted above are typically not kept for extended lengths of time). The first and second image modalities are FFDM and DBT in some embodiments.

Tissue density of the body part is estimated from the at least one medical image using a deep-learning model pretrained using images from the first imaging modality and images from the second imaging modality at 204. In the example embodiment, the deep learning model is a convolutional neural network. In other embodiments, the deep-learning model may be any supervised learning model, any self-supervised learning model, or any other deep-learning model suitable for training and operation as described herein. The deep learning model may be stored in the memory 106 of the computing device 102, may be stored in the remote device 112, or in any other suitable location accessible by the computing device 102. The received at least medical image is input to the pretrained deep-learning model and the deep-learning model outputs an estimate of the tissue density of the body part based on the at least one image. In the example embodiment, the estimate determined by the deep-learning model is a continuous numerical output. In other embodiments, the deep-learning model directly estimates tissue density according to a known assessment scheme, such as according to the assessment described in the Breast Imaging Reporting and Data System (BI-RADS) for breast tissue density.

Before being used to estimate the tissue density of the body part, the deep-learning model is trained using first imaging modality images and second modality images (i.e., it is pretrained). In the example embodiment, the training images are processed or “for presentation,” rather than the raw image data or “for process” images. In an example embodiment, the deep learning model is trained using a transfer learning technique, though any other suitable techniques to train the deep-learning model with images from the first and second imaging modalities may be used. In the example embodiment, the deep-learning model is first trained on a plurality of images of the body part tissue acquired using the first imaging modality. The plurality of images includes one or more images from each of a plurality of subjects, and each subject's images have a previously determined tissue density assigned to them. Thus, the deep-learning model first learns to estimate tissue density for the body part for images (from one or more views) of the tissue of the body part acquired using the first imaging modality. In some embodiments, the first imaging modality is FFDM. As understood by those of skill in the art, training a deep-learning model establishes various weights in the deep-learning model. In this example, the weights resulting from the initial training on the first imaging modality images are then frozen and the final classification layer of the deep-learning model is replaced with a new layer based on second imaging modality images. This involves training the deep-learning model using a plurality of second imaging modality images (where, like plurality of first imaging modality images, the the plurality includes one or more images from each of a plurality of subjects, and each subject's images have a previously determined tissue density assigned to them) with a low learning rate (e.g. a learning rate of 10⁻⁵) to adapt the weights from the first imaging modality training to the second imaging modality images. This allows the deep-learning model to retain the learned features from the first imaging modality training while adjusting to the specific characteristics of images from the second imaging modality. In some embodiments, the second imaging modality is DBT.

The result of the two trainings is a deep-learning model that is pretrained to estimate tissue density of the body part of a subject from an input of one or more are processed or “for presentation” images of the tissue of the body part of the subject acquired using either the first imaging modality or the second imaging modality. This improves the usefulness of the deep-learning model over some known systems that only function for one imaging modality and/or require knowledge of which imaging modality was used and trained for. When trained on the two imaging modalities used for nearly all breast tissue imaging, for example, the deep-learning model pretrained according to the present disclosure can estimate breast tissue density from almost any breast tissue images that are likely to be acquired, without a user needing to change any settings, input any selections of image type, determine which deep-learning model to use for the particular images available, etc. Moreover, the pretrained deep-learning model may provide more consistent results with less variability than determination made by different humans or at different times.

At 206, an estimate of tissue density of the body part of the subject based on the tissue density estimated by the deep-learning model is output. In embodiments in which the output from the deep-learning model is a continuous measurement, the output may be the determined measurement, or the measurement may be converted to any other suitable representation of tissue density. In some embodiments, the measurement is used to determine a BI-RADS category applicable to the determined measurement, and the system outputs the determined BI-RADS category instead or in addition to the raw measurement. In some embodiments, the BI-RADS categories are determined from three model-defined cut points, such as (0, 1.5) for BI-RADS A, (1.501, 2.3) for BI-RADS B, (2.301, 3.3) for BI-RADS C, and (3.301, ∞) for BI-RADS D. In still other embodiments, the system determines between “dense” and “non-dense” for the tissue density. In some embodiments, these two categories may correspond to BI-RADS A and B and BI-RADS C and D, respectively.

The output of the estimate of tissue density may be a human cognizable output, such as a display on a display device visible to the user of the system, printing the estimate on paper, etc. or may be an output that is not human cognizable, such as an electronic/digital output (e.g., storing the estimate in a memory, transmitting it to another computing device/storage device, or the like).

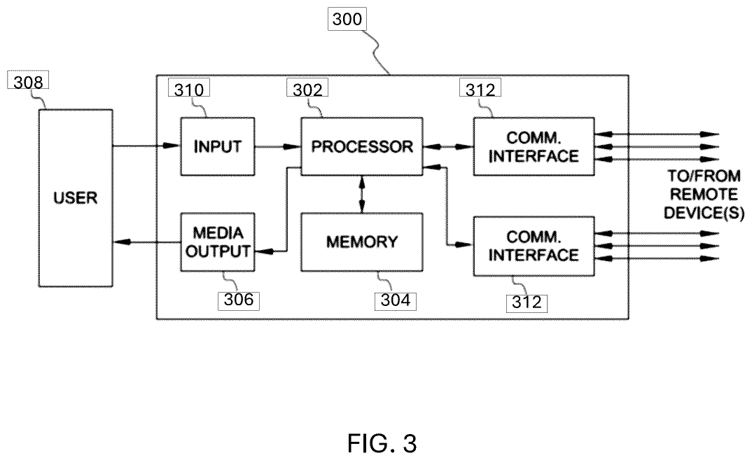

Turning to FIG. 3, an example configuration of a computing device 300 that may be used as or as part of the computing device 102, remote device 112, and/or any other computers, computing device, controllers, or the like described herein is shown. The computing device 300 includes a processor 302, a memory 304, a media output component 306, an input device 310, and communications interfaces 312. Other embodiments include different components, additional components, and/or do not include all components shown in FIG. 3.

The processor 302 is configured for executing instructions. In some embodiments, executable instructions are stored in the memory 304. The processor 302 may include one or more processing units (e.g., in a multi-core configuration). As used herein, the term “processor” refers not only to integrated circuits, but also to a controller, a microcontroller, a microcomputer, a programmable logic controller (PLC), an application-specific integrated circuit, a graphic processing unit, and other programmable circuits. The memory 304 may generally be or include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable non-transitory memory elements and is generally any device allowing information such as executable instructions and/or other data to be stored and retrieved. Such memory 304 may generally be configured to store suitable computer-readable instructions that, when implemented by the processor 302, configure, cause, or program the computing device 300 to perform various functions described herein.

The media output component 306 is configured for presenting information to user 308. The media output component 306 is any component capable of conveying information to the user 308. In some embodiments, the media output component 306 includes an output adapter such as a video adapter and/or an audio adapter. The output adapter is operatively connected to the processor 302 and operatively connectable to an output device such as a display device (e.g., a liquid crystal display (LCD), organic light emitting diode (OLED) display, cathode ray tube (CRT), “electronic ink” display, one or more light emitting diodes (LEDs)) or an audio output device (e.g., a speaker or headphones).

The computing device 300 includes, or is connected to, the input device 310 for receiving input from the user 308. The input device is any device that permits the computing device 300 to receive analog and/or digital commands, instructions, or other inputs from the user 308, including visual, audio, touch, button presses, stylus taps, etc. The input device 310 may include, for example, a variable resistor, an input dial, a keyboard/keypad, a pointing device, a mouse, a stylus, a touch sensitive panel (e.g., a touch pad or a touch screen), a gyroscope, an accelerometer, a position detector, or an audio input device. A single component such as a touch screen may function as both an output device of the media output component 306 and the input device 310.

The communication interfaces 312 enable the computing device 300 to communicate with remote devices and systems, such as allowing communication between the controller 108 and the imaging device 110, remote computing device 112, remote servers (not shown), and the like. The communication interfaces 312 may be wired or wireless communications interfaces that permit the computing device to communicate with the remote devices and systems directly or via a network, such as network 114. Wireless communication interfaces 312 may include a radio frequency (RF) transceiver, a Bluetooth® adapter, a Wi-Fi transceiver, a ZigBee® transceiver, a near field communication (NFC) transceiver, an infrared (IR) transceiver, and/or any other device and communication protocol for wireless communication. (Bluetooth is a registered trademark of Bluetooth Special Interest Group of Kirkland, Washington; ZigBee is a registered trademark of the ZigBee Alliance of San Ramon, California.) Wired communication interfaces 312 may use any suitable wired communication protocol for direct communication including, without limitation, USB, RS232, I2C, SPI, analog, and proprietary I/O protocols. In some embodiments, the wired communication interfaces 312 include a wired network adapter allowing the computing device to be coupled to a network, such as the Internet, a local area network (LAN), a wide area network (WAN), a mesh network, and/or any other network to communicate with remote devices and systems via the network. Although two communication devices 312 are shown, the computing device 300 may include more or fewer computing devices.

It should be understood that in some embodiments the computing device 300 does not include or use an input 310 or a media output 306 and a user 308 may not directly interact with the computing device. Rather, the user 308 (or another computing device) may only interact remotely with computing device 300 through the communication interface 312.

Moreover, in some embodiments the computing device 300, or parts thereof, may not be a physical computing device local to the user 308, but instead is cloud based. Thus, for example, the computing device 145 may be a cloud-based computing device or may be a physical computing device 300 using cloud-based storage for all or part of its memory 304, using cloud-based processing instead of local processing for some or all of its processing, or the like. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. As used herein, the term “cloud computing” and related terms, e.g., “cloud computing devices” refers generally to a computer architecture allowing for the use of multiple heterogeneous computing devices for data storage, retrieval, and processing. The heterogeneous computing devices may use a common network or a plurality of networks so that some computing devices are in networked communication with one another over a common network but not all computing devices. In other words, a plurality of networks may be used to facilitate the communication between and coordination of all computing devices.

In some embodiments, the computing device 300 may be embodied on or may include a desktop computer, a laptop computer, a tablet computer, a mobile phone, a microcontroller, a single board computer, or any other device operable to function as described herein.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMS (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods.

The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics and animal behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regressions, random forest classifiers, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Example

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes s for its practice. However, those of skill in the art should, in light of the present disclosure, appreciate that many changes can be made in the specific embodiments that are disclosed and still obtain a like or similar result without departing from the spirit and scope of the present disclosure.

### Example—Automated Breast Density Assessment for Full-Field Digital Mammography and Digital Breast Tomosynthesis

Mammographic density is a strong risk factor for breast cancer (BC) and is reported clinically as part of Breast Imaging Reporting and Data System (BI-RADS) results issued by radiologists. Automated assessment of density is needed that can be used for both full-field digital mammography (FFDM) and digital breast tomosynthesis (DBT) as both types of exams are acquired in standard clinical practice. A deep learning model was trained to automate the estimation of BI-RADS density from a prospective Washington University (WashU) clinic-based cohort of 9,714 women, entering into the cohort in 2013 with follow-up through, Oct. 31, 2020. The cohort included 27% non-Hispanic Black women. The trained algorithm was assessed in an external validation cohort that included 18, 360 women screened at Emory from Jan. 1, 2013 and followed through Dec. 31, 2020 that included 42% non-Hispanic Black women. The trained model-estimated BI-RADS density demonstrated substantial agreement with the density as assessed by radiologist. In the external validation, the agreement with radiologists for category B 81% and C 77% for FFDM and B 83% and C 74% for DBT show important distinction for separation of women with dense breast. A Cohen's κ of 0.72 (95% CI, 0.71, 0.73) in FFDM and 0.71 (95% CI 0.69, 0.73) in DBT was obtained. Thus, a consistent and fully automated BI-RADS estimation for both FFDM and DBT is provided using a deep learning model. The software can be easily implemented nearly anywhere for clinical use and risk prediction. T deep learning algorithm can be directly applied to processed or “for-presentation” FFDM and DBT images that are used for image interpretation in clinical practice.

### Materials and Methods

### Analytic Data Set

The Joanne Knight Breast Health Cohort at Washington University (WashU cohort) is used as the source for training data in this study. This cohort of women were recruited from November 2008 to April 2012 through an American College of Radiology (ACR) accredited and designated comprehensive breast imaging center providing routine breast screening in St. Louis and includes 10, 481 women free from breast cancer with 27% non-Hispanic Black women. The age at entry ranges from 23.1 to 93.3 where 61% of the women are postmenopausal. Eligibility criteria included consent for follow-up and attending a routine screening visit. 389 women whose entry examination led to the diagnosis of breast cancer, and 121 women whose retrieved images did not contain all four standard views were excluded. In 2015, the breast health service transitioned to all screening mammograms being DBT. All mammograms were uniformly processed using Hologic machines. For this analysis, the FFDM mammograms were restricted to be past 2013 to ensure that the recorded BI-RADS density used the 5th edition definitions and identified 9, 714 women. From this cohort, women free from cancer at their first available DBT examination (4,736 women) past 2015 were identified. On entry to the cohort, women self-reported breast cancer risk factors using established and validated measures.

### External Validation Cohort

The external validation data set is drawn from the Emory Breast Imaging Dataset (EMBED) with 116,902 patients with up to 8 years of mammograms. The public access cohort represents a 20% random sample from the full EMBED with de-identified mammograms of 22, 383 diverse women (42% non-Hispanic Black) undergoing screening or diagnostic mammograms from January 2013 through December 2020. The age at entry ranges from 20.2 to 89. Similar to the WashU cohort, women that diagnostic images (n=2, 734) and those that did not contain all four standard views (n=1,289) were excluded, leaving a cohort of 18,360 women. Approximately 35.9% (n=6,586) of the women underwent digital breast tomosynthesis (DBT), of which 58.5% (n=3,855) of these women have both FFDM and DBT at different breast screening visits included in the EMBED. The data included age, race, and time from initial digital screening mammogram to breast cancer diagnosis. Mammograms were obtained using Hologic machines (92%), GE (6%), and Fujifilm (2%). All BI-RADS density recorded in Emory uses the 5th edition.

### Training the Density Assessment Model

### Model Training in FFDM

DICOM files from presentation view were transformed into 16-bit PNG files using the pydicom and PIL tool. In the training dataset, the images are all processed by Hologic. In the external dataset, the images are processed by on Hologic machines (92%), GE (6%), and Fujifilm (2%). The trained model takes mediolateral oblique (MLO) and craniocaudal (CC) images as input. For FFDM images, these are the standard 4 view mammograms. All mammograms were each resized to 1664×2048 pixels in this analysis. All mammograms were de-meaned (centered) and normalized in a column-wise fashion. The mean and standard deviation were saved from the training dataset and subsequently applied onto the external validation.

Each view of the mammogram was independently encoded by using ResNet-18 with a global max pooling layer to compress the image representation to a 512-dimensional vector. The model was trained using the Adam optimizer with a learning rate of 10-4 and a weight decay of 10-5. Given the 4-views for each woman, a 2048-dimensional vector resulted that summarizes all information embedded in the mammograms. Results are reported for the epoch that had the lowest cross entropy loss on the validation set.

### Transfer Learning for Synthetic DBT

Similar preprocessing procedure were performed in synthetic DBT images. Specifically, the synthesized DBT that are automatically generated from series of raw 2D projections were used. All synthesized DBT were each resized to 1664×2048 pixels in this analysis. All synthesized DBT were de-meaned (centered) and normalized in a column-wise fashion. The mean and standard deviation were saved from the training dataset and subsequently applied onto the external validation.

Transfer learning is a powerful technique in machine learning that involves leveraging a pre-trained model on a new, but related, task. Here, the weights from previously trained model using the FFDM were frozen and the final classification layer of the pre-trained model was replaced with a new layer in the synthetic DBT. This involved training the model with a low learning rate of 10-5 to adapt the weights from the FFDM to the synthetic DBT. Fine-tuning allows the model to retain the learned features from the FFDM while adjusting to the specific characteristics of synthetic DBT images.

### Density Output

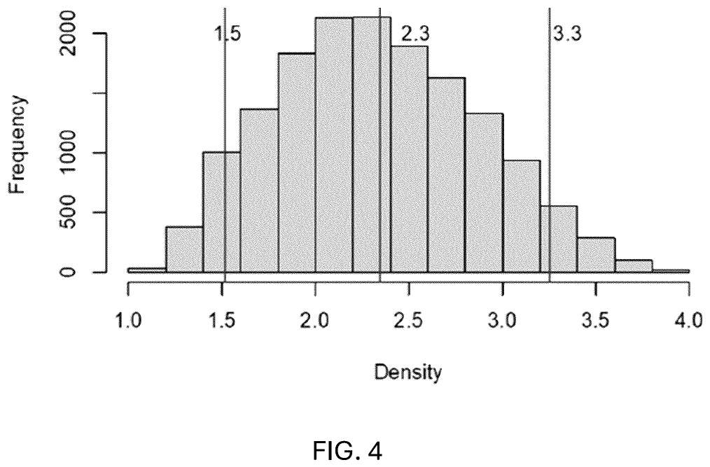

The output from the model is continuous measure that is converted into the BI-RADS categories. The BI-RADS categories are determined from three model-defined cut points. The model-defined cut-off points are (0, 1.5) for BI-RADS A, (1.501, 2.3) for BI-RADS B, (2.301, 3.3) for BI-RADS C, and (3.301, ∞) for BI-RADS D. This cut-off is agnostic to FFDM or synthetic DBT. For illustration, FIG. 4 is a graph of the distribution of the continuous density measures estimated from the FFDM using the proposed model in the external validation cohort.

### Statistical Analysis

Two deep learning models were developed using the WashU cohort to estimate BI-RADS density at each of the examinations. The model takes all four views of processed or “for presentation” mammograms (craniocaudal [CC] and mediolateral-oblique [MLO] views) as input. The first model was trained using only FFDM mammograms. The second model was fine-tuned from the FFDM model to accommodate synthetic DBT that are generated from the series of raw projections as described above. This model for synthetic DBT uses a transfer learning approach that transfers knowledge from the pre-trained FFDM model on the new synthetic DBT task.

### Testing and Validation

The WashU dataset was randomly split with 20% of women in testing, 15% in validation, and the rest for training. To assess the classification performance of the proposed algorithm within the WashU cohort, the confusion matrix was generated for the 20% of women in the testing set. The Emory Cohort was only used for testing. Therefore, all women within the Emory cohort that have been constructed have been projected back onto the trained model in the WashU cohort to record the model performance. Model performance is reported using confusion matrix that compares the absolute counts of radiologist scored BI-RADS density vs. BI-RADS estimated via the proposed method. The evaluation of the concordance between radiologists' BI-RADS scores and the BI-RADS density estimated by the proposed method was measured using Cohen's κ. Mis-classification error is reported with a confusion matrix that compares the absolute counts of radiologist scored BI-RADS density vs. BI-RADS estimated via the proposed method. The confusion matrix shows BI-RADS A, B, C, D as well as by dense (A/B) vs. non-dense (C/D).

Additionally, concordance between radiologists' assessments using BI-RADS (5th edition) and the BI-RADS density estimated by the proposed method was evaluated. Cohen's κ is a measure used to quantify inter-rater agreements. If the raters are in complete agreement, then κ=1; if there is no agreement, then κ=0. Performances for both the mis-classification error and inter-rater agreement are separately reported for FFDM and synthetic DBT.

### Results

### Cohort Characteristics

In the WashU cohort, breast cancer risk factors were assessed at entry to the cohort for the women in this prospective study, as shown in Table 1 below. There was no important difference between FFDM and synthetic DBT distribution in the qualitative breast density assigned by the radiologist (5th edition BI-RADS A/B categories [“not dense”] vs BI-RADS C/D categories [“dense”]). The cohort included 26% non-Hispanic Black and 70% non-Hispanic White women.

TABLE 1

| | WashU derivation cohort | | | | Emory validation cohort | | | |
|---|---|---|---|---|---|---|---|---|
| | FFDM | | DBT | | FFDM | | DBT | |
| | (n = 9,714) | | (n = 4,736) | | (n = 15,629) | | (n = 6,586) | |
| Mean (sd) | | | | | | | | |
| Age (years) | 55.7 | (10.0) | 54.6 | (8.7) | 55.6 | (12.2) | 57.8 | (11.6) |
| Number (%) | | | | | | | | |
| BI-RADS | | | | | | | | |
| A | 999 | (10.3%) | 454 | (9.6%) | 1647 | (10.6%) | 720 | (10.9%) |
| B | 4926 | (50.8%) | 2323 | (49.0%) | 6488 | (41.5%) | 2628 | (39.9%) |
| C | 3350 | (34.5%) | 1705 | (36.1%) | 6565 | (42.0%) | 2825 | (42.9%) |
| D | 422 | (4.4%) | 239 | (5.0%) | 926 | (5.9%) | 413 | (6.3%) |
| NR | 0 | (0%) | 15 | (0.3%) | 0 | (0%) | 0 | (0%) |
| Race | | | | | | | | |
| White | 6768 | (69.8%) | 3321 | (70.1%) | 6413 | (41.1%) | 2560 | (38.9%) |
| Black | 2549 | (26.3%) | 1251 | (26.4%) | 6584 | (42.1%) | 3031 | (46.0%) |
| Asian | 83 | (0.9%) | 36 | (0.8%) | 968 | (6.2%) | 465 | (7.1%) |
| Others | 88 | (0.9%) | 34 | (0.7%) | 268 | (1.7%) | 62 | (0.9%) |
| NR | 209 | (2.1%) | 94 | (2.0%) | 1393 | (8.9%) | 468 | (7.1%) |
| Breast cancer cases | 469 | (4.8%) | 105 | (2.2%) | 408 | (2.6%) | 133 | (2.0%) |

Comparable BI-RADS distribution and ethnic diversity in the Emory external validation cohort are reported in Table 1. The cohort included 42 non-Hispanic Black women. There was no important difference between FFDM and synthetic DBT distribution in the qualitative breast density assigned by the radiologist (5th edition BI-RADS A/B categories [“not dense”] vs BI-RADS C/D categories [“dense”]).

### Model Performance in FFDM

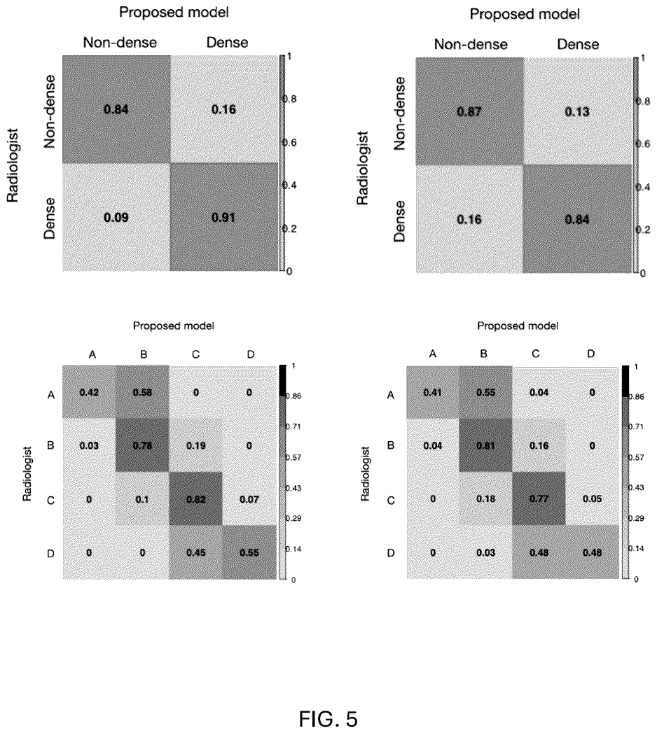

The estimated mis-classification counts against radiologists reading are shown in a confusion matrix in FIG. 5 using FFDMs. The model was first evaluated in an internal validation composed of 20% of random sample from the WashU cohort that was left out from the training data. The BI-RADS classification as predicted by the proposed model exhibits close agreement with the radiologist scoring. As seen in the top left matrix, the model agrees with the radiologist 84% of time for women with non-dense (BI-RADS A/B) breast and 91% of the time for women with dense breast (BI-RADS C/D). The confusion matrix separated for the 4 categories of BI-RADS are also displayed in the bottom left matrix. This resulted in a Cohen's κ of 0.74 (95% CI, 0.73, 0.75) for the inter-rater agreements using the 4 categories in the WashU cohort.

Similarly, when evaluating performance in the external validation cohort in Emory, the proposed model estimated BI-RADS density, top right matrix, agrees with the radiologist 87% of time for women with non-dense (BI-RADS A/B) breast and 84% of the time for women with dense breast (BI-RADS C/D). The confusion matrix separated for the 4 categories of BI-RADS are also displayed in the bottom right matrix. Importantly the category B and C had high agreement with radiologists (B 81% and C 77%) in the external validation. This resulted in a Cohen's κ of 0.72 (95% CI, 0.71, 0.73) for the inter-rater agreements using the 4 categories in the external validation cohort.

### Model Performance in Synthetic DBT

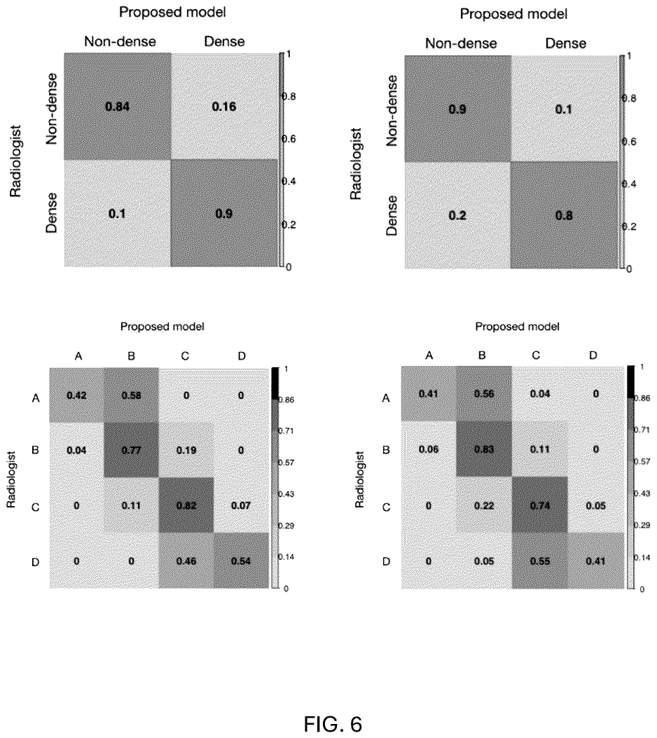

The estimated mis-classification counts are shown in a confusion matrix in FIG. 6 when using the synthetic DBT images. Similar performances here are seen when compared to FFDM. In the WashU internal validation, the model agrees with the radiologist 84% of time for women with non-dense (BI-RADS A/B) breast and 90% of the time for women with dense breast (BI-RADS C/D) as seen in the upper left matrix. The separate results for 4 categories are displayed in the bottom left matrix, resulting in a Cohen's κ of 0.74 (95% CI, 0.73, 0.75).

When evaluated in the external cohort, the proposed model estimated BI-RADS density agrees with the radiologist 90% of time for women with non-dense (BI-RADS A/B) breast and 80% of the time for women with dense breast (BI-RADS C/D) as seen in the upper right matrix. Importantly, in the 4-category setting, the category B and C had high agreement with radiologists (B 83% and C 74%) in the external validation. The confusion matrix separated for the 4 categories of BI-RADS are displayed in the bottom right matrix, resulting in a Cohen's κ of 0.71 (95% CI 0.69, 0.73).

### Subgroup Analysis

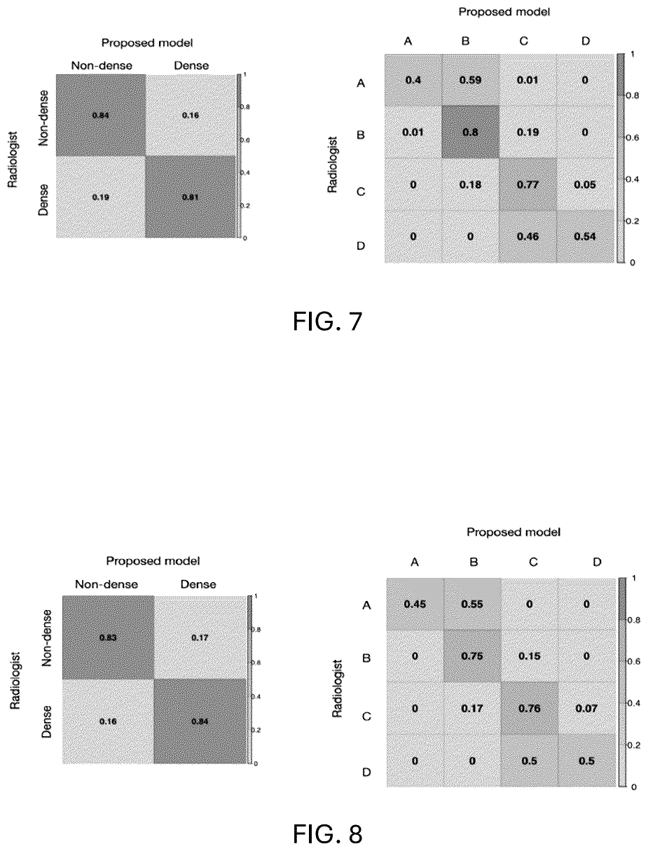

Results in the external validation dataset for the subset of women who were diagnosed with breast cancer in their follow up since baseline are shown in FIGS. 7 and 8. FIG. 7 shows the results under FFDM and FIG. 8 demonstrates the results under synthetic DBT.

### Discussion

With the widespread use of DBT in the US, it would be beneficial for automated density measures to be readily available. The disclosed automated tool can assess mammographic density in both FFDM and synthetic DBT mammography. The deep learning algorithm calibrates well with the radiologist rating in BI-RADS in both the internal validation and the independent external validation that is racially diverse with 42% of Non-Hispanic Black women. There are strong agreements between the deep learning algorithm with radiologists when looking at dense vs. non-dense in the external validation. When looking at 4 categories of density, the Cohen's κ was 0.71-0.72 in FFDM and synthetic DBT for the inter-rater agreements in the external validation cohort, is stronger than reported in other studies. Thus, this study extends beyond previous work, where the women were largely limited to NH White women.

The trained algorithm has advantages over existing mammographic density estimation tools that function for both FFDM and DBT. For instance, the disclosed model does not require raw or “for process” images as input. Most institutions do not store those for more than a month, meaning that exams cannot be subsequently reprocessed after acquisition if raw or “for process” images were required as input. The trained deep-learning model of this disclosure uses processed or “for-presentation” FFDM and synthetic DBT that are typically, permanently archived. This algorithm may tie into routine breast imaging services and deliver output of density and BIRADS category to the reading radiologist as an aid to classifying density which is now a reportable feature in the US, similar to other programs in use and aims to reduce variability among providers over time.

In the external validation, a Cohen's κ of 0.72 (95% CI, 0.71, 0.73) for FFDM and 0.71 (95% CI 0.69, 0.73) was obtained for synthetic DBTs. When comparing inter-rater agreements of FFDM with some known systems, one known system reported a Cohen's κ of 0.57 (95% CI, 0.55, 0.59) and a second reported a k of 0.46 (95% CI, 0.44, 0.47). Additionally, BI-RADS density output by the present system is achieved by grouping a continuous measure of density estimated from the proposed algorithm. Such a continuous measure may be more sensitive when studying changes in density over time.

FIG. 1. Comparison of density estimation (BI-RADS density 5th edition) by deep learning model and by radiologists reading Full Field Digital Mammograms (FFDM). Left=WashU (n=1, 943); Right=Emory (n=15, 629)). The internal training data was excluded from the results represented in WashU.

FIG. 2. Comparison of density estimation (BI-RADS density 5th edition) by deep learning model and by radiologists reading Digital Breast Tomosynthesis (DBT) using synthetic DBT. Left=WashU (n=945); Right=Emory (n=6,586). The internal training data was excluded from the results represented in WashU.

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “have” and “comprise,” “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A computer implemented method of estimating tissue density of a body part of a subject from a medical image of tissue of the body part of the subject, the method comprising:
receiving at least one medical image of tissue of the body part of the subject, the received medical image being from a first imaging modality or a second imaging modality;
estimating tissue density of the body part from the at least one medical image using a deep-learning model pretrained using images from the first imaging modality and images from the second imaging modality; and
outputting an estimate of tissue density of the body part of the subject based on the tissue density estimated by the deep-learning model.

2. The method of claim 1, wherein the body part comprises a breast and the tissue density of the body part comprises breast tissue density.

3. The method of claim 2, wherein the first imaging modality is full-field digital mammography (FFDM).

4. The method of claim 3, wherein the second imaging modality is digital breast tomosynthesis (DBT).

5. The method of claim 1, further comprising pretraining the deep-learning model using images from the first imaging modality and the second imaging modality.

6. The method of claim 5, wherein pretraining the deep-learning model comprises training the deep-learning model first using only images from the first imaging modality and refining, using only images from the second imaging modality, training of the deep-learning model that was already trained using images from the first imaging modality.

7. The method of claim 6, wherein refining training of the deep-learning model that was already trained using images from the first imaging modality comprises refining the deep-learning model using transfer learning.

8. The method of claim 1, wherein outputting the estimate of tissue density comprises determining a Breast Imaging Reporting and Data System (BI-RADS) category applicable to the estimated tissue density from the deep-learning model and outputting the determined BI-RADS category.

9. The method of claim 1, further comprising controlling an imaging system to generate the at least one medical image of tissue of the body part of the subject.

10. The method of claim 1, wherein receiving the at least one medical image of tissue of the body part of the subject comprises receiving a plurality of images presenting processed or for presentation views of tissue of the body part of the subject.

11. A system for estimating breast tissue density of a subject from a medical image of tissue of a breast of the subject, the system comprising a computing device having a processor and a memory, the memory storing instructions that cause the processor to:
receive at least one medical image of tissue of the breast of the subject, the received medical image being an image from a first imaging modality or a second imaging modality;
estimate breast tissue density from the at least one medical image using a deep-learning model pretrained using images from the first imaging modality and images from the second imaging modality; and
output an estimate of the breast tissue density of the subject.

12. The system of claim 11, wherein the first imaging modality is full-field digital mammography (FFDM) and the second imaging modality is digital breast tomosynthesis (DBT).

13. The system of claim 11, further comprising an imaging system of the first imaging modality or the second imaging modality, wherein the memory stores instructions that further cause the processor to control the imaging system to generate the at least one medical image of tissue of the breast of the subject.

14. The system of claim 11, wherein memory stores instructions that cause the processor to output the estimate of tissue density by determining a Breast Imaging Reporting and Data System (BI-RADS) category applicable to the estimated tissue density from the deep-learning model and outputting the determined BI-RADS category.

15. The system of claim 11, wherein the instructions further program the processor to pretraining the deep-learning model using a plurality of images from the first imaging modality and the second imaging modality by training the deep-learning model first using only images from the first imaging modality and subsequently refining, using only images from the second imaging modality, training of the deep-learning model that was already trained using images from the first imaging modality.

16. A method of estimating breast tissue density of a subject from at least one medical image of tissue of a breast of the subject, the method comprising:
receiving a plurality of first images acquired using a first imaging modality, the plurality of first images including at least one image of breast tissue for each first subject of a plurality of first subjects and a breast tissue density of each first subject having been previously determined;
training a deep-learning model on the first plurality of images and the previously determined breast tissue density for each first subject;
receiving a plurality of second images acquired using a second imaging modality, the plurality of second images including at least one image of breast tissue for each second subject of a plurality of second subjects and a breast tissue density of each second subject having been previously determined;
further training the deep-learning model on the second plurality of images and the previously determined breast tissue density for each second subject;
receiving at least one medical image of tissue of the breast of the subject;
inputting the at least one medical image of tissue of the breast of the subject into the further trained deep-learning model; and
receiving an estimated breast tissue density for the subject output by the further trained deep-learning model.

17. The method of claim 16, wherein the first imaging modality is full-field digital mammography (FFDM) and the second imaging modality is digital breast tomosynthesis (DBT).

18. The method of claim 16, wherein further training the deep-learning model on the second plurality of images and the previously determined breast tissue density for each second subject comprises freezing a plurality of weights in the deep learning model after training the deep-learning model on the first plurality of images and the previously determined breast tissue density for each first subject, and replacing a final classification layer of the deep-learning model with a new layer based on the second plurality of images and the previously determined breast tissue density for each second subject.

19. The method of claim 16, further comprising:
determining a Breast Imaging Reporting and Data System (BI-RADS) category applicable to the estimated tissue density from the deep-learning model; and
outputting the determined BI-RADS category.

20. The method of claim 16, further comprising controlling an imaging system to generate the at least one medical image of tissue of the breast of the subject.
